(19) United States
(12) Patent Application Publication
(10) Pub. No.: US 2024/0165107 A1
(43) Pub. Date: May 23, 2024

(54) Long Acting Injectable Compositions of Cariprazine or its Pharmaceutically Acceptable Salts

(71) Applicant: Cipla Limited, Mumbai (IN)

(72) Inventors: Pradeep BHADAURIA, Weston, FL (US); Tushar RAJYAGURU, Bhavnagar (IN); Abhinesh KUMAR, Ahmedabad (IN); Harshad HARDE, Nashik (IN); Mukesh KUMAR, Navi Mumbai (IN); Manoj Mahadu WALUNJ, Ahmednagar (IN); Arnab Kumar SINGHADEO, Hooghly (IN)

(21) Appl. No.: 18/282,007

(22) PCT Filed: Mar. 15, 2022
(86) PCT No.: PCT/IN2022/050242

§ 371 Date: Sep. 14, 2023

(30) Foreign Application Priority Data
Mar. 16, 2021 (IN) 202121011063

(51) Int. Cl. A61K 31/495; A61K 9/00; A61K 47/10

(52) U.S. Cl. CPC A61K 31/495; A61K 9/0019; A61K 47/10

(57) ABSTRACT

The present invention discloses to a long acting injectable composition comprising cariprazine or its pharmaceutically acceptable salts thereof and process to prepare the same. The present invention also discloses to use of the long acting injectable composition comprising cariprazine or its pharmaceutically acceptable salts thereof in the treatment of psychotic disorders.

### CROSS-REFERENCE TO RELATED APPLICATIONS

This application claims priority to Indian Provisional Patent Application No. 202121011063, filed 16 ^{th} March 2021 which is incorporated herein in its entirety.

### FIELD OF INVENTION

The present invention relates to a long acting injectable composition comprising cariprazine or its pharmaceutically acceptable salts thereof and process to prepare the same. The present invention also relates to use of the long acting injectable composition comprising cariprazine or its pharmaceutically acceptable salts thereof in the treatment of psychotic disorders.

### BACKGROUND OF INVENTION

Cariprazine is an atypical antipsychotic acting through a combination of partial agonist activity at central dopamine D2 and serotonin 5-HT1A receptors and antagonist activity at serotonin 5-HT2A receptors. Cariprazine acts as a partial agonist at the dopamine D3 and D2 receptors with high binding affinity and at the serotonin 5-HT 1A receptors. Cariprazine acts as an antagonist at 5-HT2B and 5-HT2A receptors with high and moderate binding affinity as well as it binds to the histamine H1 receptors. Cariprazine shows lower binding affinity to the serotonin 5-HT2C and alA- adrenergic receptors and has no appreciable affinity for cholinergic muscarinic receptors.

Cariprazine is approved in US as immediate release oral capsules under the brand name Vraylar. Cariprazine was first disclosed in WO2005012266 and use in psychoses (e.g. schizophrenia, schizo-affective disorders, etc.), drug (e.g. alcohol, cocaine and nicotine, opioids, etc.) abuse, cognitive impairment accompanying schizophrenia, mild-to-moderate cognitive deficits, dementia, psychotic states associated with dementia, eating disorders (e.g. bulimia nervosa, etc.), attention deficit disorders, hyperactivity disorders in children, psychotic depression, mania, paranoid and delusional disorders, dyskinetic disorders (e.g. Parkinson's disease, neuroleptic induced parkinsonism, tardive dyskinesias) anxiety, sexual dysfunction, sleep disorders, emesis, aggression, autism.

WO2010009309 disclose immediate release solid oral compositions of cariprazine. WO2009104739 discloses lactose containing immediate release solid oral cariprazine compositions. Another immediate release granule composition containing cariprazine is disclosed in WO2017178999. All the composition described in these references have to be dosed once on a daily basis.

There is a need for less frequent administration for patients especially with a longer treatment period. In addition, a significant subset of the target population is likely to be patients that are both elderly and forgetful. Such patients often require time consuming and expensive supervised administration. Reduction in dosing frequency offers significant advantages by reducing the indirect human cost of drug treatment, improved patient compliance as well as reducing medical practitioners' time required for supervised drug administration.

WO2018229641 discloses oral pharmaceutical compositions and methods for the modified release delivery of cariprazine. However, evidences of abdominal discomfort, abdominal pain, abdominal pain lower, abdominal pain upper, gastrointestinal pain have been observed with oral administration of cariprazine. EMEA assessment report for immediate release cariprazine capsules indicate that the oral absolute bioavailability was 52-63% in rats and — 64-80% dogs, indicating incomplete absorption and some first-pass effect. Administration of drugs by parenteral route have shown to achieve precise control on blood levels and also reduced adverse effects observed upon oral administration.

There is a need for an injectable dosage form of cariprazine which addresses the adverse effects observed by oral administration. The present invention provides a long acting injectable composition comprising cariprazine or its pharmaceutically acceptable salts thereof which allows for a reduced dosing frequency with lower adverse effects.

### OBJECT OF THE INVENTION

An object of the present invention is to provide a long acting injectable composition comprising cariprazine or its pharmaceutically acceptable salts thereof.

Another object of the present invention is use of a long acting injectable composition comprising cariprazine or its pharmaceutically acceptable salts thereof in the treatment of psychotic disorders.

One more object of the present invention is to provide a long acting injectable composition comprising cariprazine or its pharmaceutically acceptable salts thereof in the form of an aqueous suspension.

Yet another object of the present invention is to provide a long acting injectable composition comprising cariprazine pharmaceutically acceptable salts in the form of a depot composition which forms an in-situ gel upon injection.

A further object of the present invention is to provide a long acting injectable composition comprising cariprazine pharmaceutically acceptable salts in the form of an implant.

It is also an object of the present invention to provide a long acting injectable composition comprising cariprazine pharmaceutically acceptable salts in the form of microspheres.

One more object of the present invention is to provide a long acting injectable composition comprising cariprazine pharmaceutically acceptable salts thereof in the form of an oil based depot composition.

### SUMMARY OF THE INVENTION

The present invention relates to a long acting injectable composition comprising cariprazine or its pharmaceutically acceptable salts thereof and process to prepare the same. The present invention also relates to use of the long acting injectable composition comprising cariprazine or its pharmaceutically acceptable salts thereof in the treatment of psychotic disorders.

In one embodiment, the long acting injectable composition comprising cariprazine or its pharmaceutically acceptable salts thereof is an aqueous suspension.

In another embodiment, the long acting injectable composition comprises cariprazine or its pharmaceutically acceptable salts thereof, a suspending agent, a preservative, a buffer, a tonicity adjusting agent and vehicle.

In one embodiment, the long acting injectable composition comprises cariprazine or its pharmaceutically acceptable salts thereof, a suspending agent, a buffer, and vehicle, wherein the injectable composition is in the form of an aqueous suspension.

In one more embodiment, the long acting injectable composition comprises cariprazine or its pharmaceutically acceptable salts thereof, polyethylene glycol, monobasic sodium phosphate and dibasic sodium phosphate as buffer, polysorbate 80, sodium chloride and water for injection.

In yet another embodiment, the long acting injectable composition comprises cariprazine or its pharmaceutically acceptable salts thereof, polyethylene glycol, monobasic sodium phosphate and dibasic sodium phosphate as buffer, Sorbitan mono-oleate, sodium chloride and water for injection.

In a further embodiment, the long acting injectable composition comprises cariprazine or its pharmaceutically acceptable salts thereof, polyethylene glycol, monobasic sodium phosphate and dibasic sodium phosphate as buffer, polysorbate 80, Sorbitan mono-oleate, sodium chloride and water for injection.

In one embodiment, the long acting injectable composition comprises cariprazine base, polyethylene glycol 4000, sorbitan monolaurate, polysorbate 20, citric acid monohydrate, sodium dihydrogen phosphate monohydrate, and water for injection. In another embodiment, the long acting injectable composition comprises cariprazine base, polyethylene glycol 4000, polysorbate 20, citric acid monohydrate, sodium dihydrogen phosphate monohydrate, and water for injection.

In an embodiment, the long acting injectable composition comprising cariprazine or its pharmaceutically acceptable salts thereof is a depot composition which forms an in-situ gel upon injection.

In another embodiment, the long acting injectable composition comprises cariprazine or its pharmaceutically acceptable salts thereof, atleast one rate controlling polymer, and solvents, wherein the said composition is an in-situ gelling composition.

In a further embodiment, the long acting injectable composition comprises cariprazine or its pharmaceutically acceptable salts thereof, atleast one rate controlling polymer comprising poly(lactic-co-glycolic acid) polymer, sucrose acetate isobutyrate, triethyleneglycol poly(orthoester) polymer, and atleast one solvent comprising 2-N-methyl pyrollidone, dimethyl acetamide, dimethyl sulfoxide, ethanol, glyceryl triacetate, wherein the said composition is an in-situ gelling composition.

In an embodiment, the long acting injectable composition comprises about 25 mg/ml to about 180 mg/ml cariprazine or its pharmaceutically acceptable salt thereof, about 25 mg/ml to about 500 mg/ml poly(lactic-co-glycolic acid) polymer and N-methyl pyrrolidone.

In another embodiment, the long acting injectable composition comprises about 25 mg/ml to about 180 mg/ml cariprazine or its pharmaceutically acceptable salt thereof, about 25 mg/ml to about 500 mg/ml poly(lactic-co-glycolic acid) polymer, about 50 mg/ml to about 150 mg/ml polyethylene glycol and N-methyl pyrrolidone.

In one more embodiment, the long acting injectable composition comprises about 25 mg/ml to about 180 mg/ml cariprazine or its pharmaceutically acceptable salt thereof, about 50 mg/ml to about 1000 mg/ml sucrose acetate isobutyrate polymer and N-methyl pyrrolidone.

In a further embodiment, the long acting injectable composition comprises about 25 mg/ml to about 180 mg/ml cariprazine or its pharmaceutically acceptable salt thereof, about 50 mg/ml to about 2000 mg/ml triethyleneglycol poly(orthoester) polymer and N-methyl pyrrolidone. In an embodiment, the long acting injectable composition comprising cariprazine or its pharmaceutically acceptable salts thereof is a depot composition which is in the form of an implant.

In another embodiment, the long acting injectable composition comprises cariprazine or its pharmaceutically acceptable salts thereof, and atleast one rate controlling polymer, wherein the said composition is an implant.

In yet another embodiment, the long acting injectable composition comprises cariprazine or its pharmaceutically acceptable salts thereof, and atleast one rate controlling polymer comprising poly(lactic-co-glycolic acid) polymer, polylactic acid polymers, wherein the said composition is an implant.

In one embodiment, the long acting injectable composition is an implant having a length of about 10 mm to about 30 mm and a diameter of about 0.5 mm to about 5 mm.

In a preferred embodiment, the long acting injectable composition is an implant having a length of about 15 mm and a diameter of about 1.5 mm.

In an embodiment, the long acting injectable composition comprising cariprazine or its pharmaceutically acceptable salts thereof is a microsphere composition.

In another embodiment, the long acting injectable composition comprises cariprazine or its pharmaceutically acceptable salts thereof, and atleast one rate controlling polymer, wherein the said composition is in the form of microspheres.

In yet another embodiment, the long acting injectable composition comprises cariprazine or its pharmaceutically acceptable salts thereof, and atleast one rate controlling polymer comprising poly(lactic-co-glycolic acid) polymer, polylactic acid polymers, wherein the said composition is in the form of microspheres. In one more embodiment, the long acting injectable microsphere composition may be supplied with a diluent. An exemplary embodiment of a diluent may contain carboxymethyl cellulose sodium, mannitol, polysorbate 80 and water.

In one embodiment, the long acting injectable composition comprises about 180 mg cariprazine or its pharmaceutically acceptable salts thereof and about 820 mg poly(lactic-co-glycolic acid) polymer.

In another embodiment, the long acting injectable composition comprises about 160 mg cariprazine or its pharmaceutically acceptable salts thereof and about 840 mg poly(lactic-co-glycolic acid) polymer.

In an embodiment, the long acting injectable composition comprises about 120 mg cariprazine or its pharmaceutically acceptable salts thereof and about 880 mg poly(lactic-co-glycolic acid) polymer.

In one embodiment, the long acting microsphere injectable composition can be manufactured using single emulsification, double emulsification, phase-coacervation, cross-linking, and spray drying.

In one more embodiment, the long acting injectable composition is in the form of microspheres having a particle size D90 of about 1 micron to about 200 microns.

In an embodiment, the long acting injectable composition comprising cariprazine or its pharmaceutically acceptable salts thereof is an oil based depot composition.

In one more embodiment, the long acting injectable composition comprises cariprazine or its pharmaceutically acceptable salts thereof, atleast one oil, and a preservative.

In one embodiment, the long acting injectable composition comprises cariprazine or its pharmaceutically acceptable salts thereof, sesame oil and benzyl alcohol.

In yet another embodiment, the long acting injectable composition comprises cariprazine or its pharmaceutically acceptable salts thereof, cotton seed oil and benzyl alcohol.

In one more embodiment, the long acting injectable composition is in the form of oil based depot having a particle size D90 of about 1 micron to about 200 microns.

The long acting injectable composition may contain about 5 mg to about 600 mg of cariprazine or its pharmaceutically acceptable salts thereof, such as from about 10 mg to about 400 mg, preferably about 20 mg to about 180 mg of cariprazine or its pharmaceutically acceptable salts thereof. In a preferred embodiment, the cariprazine is present as cariprazine hydrochloride.

The long acting injectable composition comprising cariprazine or its pharmaceutically acceptable salts thereof may provide a release for over a period of at least 15 days, for example over a period of 1 month or over a period of 3 months.

In one more embodiment, the long acting injectable composition comprising cariprazine or its pharmaceutically acceptable salts thereof provided herein has a long shelf life, i.e., it is stable during long term storage. The pharmaceutical composition or solution may contain greater than about 80%, such as greater than about 85%, greater than about 90%, greater than about 95% or greater than about 98% of the initial amount of cariprazine or its pharmaceutically acceptable salts thereof in the composition after being stored for 3 or 6 months or 1, 2 or 3 years at 25° C.

In an embodiment, the long acting injectable composition of the present invention have a pH of about 1 about 10. For example, the preferred pH range of the inhalation composition is about 1 to about 10, preferably about 3 to about 8, more preferably about 5 to about 7.

In one embodiment, the long acting injectable composition of the present invention has a particle size D90 of cariprazine or its pharmaceutically acceptable salt between about 1 micron to about 200 microns.

In one more embodiment, the long acting injectable composition of the present invention has a viscosity ranging from about 20 cps to about 200 cps.

In a further embodiment, the long acting injectable composition of the present invention has a sediment height of about 30% to about 80%.

In an embodiment, the redispersion time for the long acting injectable composition of the present invention is less than 1 minute, preferably about 5 seconds to about 50 seconds, more preferably about 10 seconds to about 30 seconds.

One embodiment is a long acting injectable aqueous suspension composition of the present invention having a particle size D90 between about 1 micron to about 200 microns, pH ranging from about 3 to about 8, viscosity ranging from about 20 cps to about 200 cps, sediment height between about 30% to about 80%, and redispersion time less than 1 min.

One more embodiment is a long acting injectable microsphere composition of the present invention having a particle size D90 between about 1 micron to about 200 microns, pH ranging from about 3 to about 8, viscosity ranging from about 20 cps to about 200 cps, sediment height between about 30% to about 80%, and redispersion time less than 1 min.

Another embodiment is a premeasured, prepackaged, premixed long acting injectable composition comprising cariprazine or its pharmaceutically acceptable salts thereof Preferably, the long acting injectable composition comprising cariprazine or its pharmaceutically acceptable salts thereof is a ready-to-use composition which does not require any mixing or dilution by the subject prior to administration. The long acting injectable composition comprising cariprazine or its pharmaceutically acceptable salts thereof may be administered for the treatment of psychotic disorders.

Yet another embodiment is a method of administering cariprazine or its pharmaceutically acceptable salts thereof for use in treatment of psychotic disorders.

One more embodiment is a kit comprising an injection device, instructions for using the device and the container containing the long acting injectable composition comprising cariprazine or its pharmaceutically acceptable salts thereof of the present invention.

Yet another embodiment is a kit comprising an injection device, instructions for using the device, a container containing the long acting injectable composition comprising cariprazine or its pharmaceutically acceptable salts thereof of the present invention and a separate vial containing a diluent.

Other objects, features and advantages of the present invention will be apparent to those of ordinary skill in the art in view of the following detailed description of the invention and accompanying drawings.

### DETAILED DESCRIPTION OF THE INVENTION

The present invention relates to a long acting injectable composition comprising cariprazine or its pharmaceutically acceptable salts thereof and process to prepare the same. The present invention also relates to use of the long acting injectable composition comprising cariprazine or its pharmaceutically acceptable salts thereof in the treatment of psychotic disorders.

The term “cariprazine or its pharmaceutically acceptable salts thereof” includes cariprazine base, salts, esters, solvates, hydrates, enantiomers, and polymorphs.

As used herein, the terms “a”, “an”, means one or more.

The term “injection” in the present context encompasses administration by parenteral routes like intravenous, subcutaneous, intramuscular, intrathecal, intradermal.

The term “psychotic disorders” include schizophrenia, schizoaffective disorders, cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders, attention deficit disorders, hyperactivity disorders in children, psychotic depression, mania, paranoid and delusional disorders, Parkinson's disease, neuroleptic induced parkinsonism, tardive dyskinesias, anxiety, sexual dysfunction, sleep disorders, emesis, aggression, autism.

The term “about” includes the indicated amount ±10%.

Cariprazine

Cariprazine is an atypical antipsychotic and is chemically known as trans-N-{4-[2-[4-(2,3 -dichl orophenyl)piperazine-1-yl]ethyl] cyclohexyl}-N′,N′-dimethylurea. The molecular formula for cariprazine is C₂₁ H₃₂ Cl₂ N₄O and molecular weight is 427.4 g/mol. The structure of cariprazine is as given below:

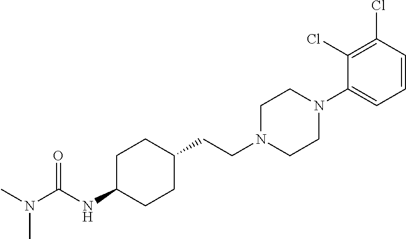 [SMILES: CN(C)C(=O)N[C@H]1CC[C@H](CCN2CCN(c3cccc(Cl)c3Cl)CC2)CC1]

The long acting injectable composition may contain about 5 mg to about 600 mg of cariprazine or its pharmaceutically acceptable salts thereof, such as from about 10 mg to about 400 mg, about 20 mg to about 180 mg, about 25 mg to about 180 mg, about 90 mg to about 180 mg of cariprazine or its pharmaceutically acceptable salts thereof.

Unless otherwise indicated herein, the term “pharmaceutically acceptable salts” refers to salts obtained by reacting cariprazine as a base with an inorganic or organic acid to form a salt, for example, salts of hydrochloric acid, sulfuric acid, phosphoric acid, methane sulfonic acid, ethane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, palmitic acid, oleic acid and carbonic acid. Pharmaceutically acceptable salts also include those in which cariprazine functions as an acid and is reacted with an appropriate base to form, e.g., sodium, potassium, calcium, magnesium, ammonium, and choline salts.

The cariprazine or its pharmaceutically acceptable salts thereof may be in micronized form. Suitable micronization techniques such as dry milling, wet milling, air jet milling, sieving, homogenizing using a homogenizer such as rotor-stator and/or high pressure homogenizer such as a microfluidizer can be used for micronization of cariprazine or its pharmaceutically acceptable salts thereof. Alternately, the cariprazine or its pharmaceutically acceptable salts thereof may be in unmicronized form.

The long acting injectable composition of the present invention may have a particle size D90 of cariprazine or its pharmaceutically acceptable salt between about 1 micron to about 200 microns.

Long acting injectable compositions The long acting injectable composition comprising cariprazine or its pharmaceutically acceptable salts thereof may be an aqueous suspension, in-situ gel depot injection, implant, microsphere or an oily depot.

The long acting injectable composition of the present invention may contain sterile cariprazine or its pharmaceutically acceptable salts thereof. Cariprazine may be sterilized by techniques known in the art such as irradiation, dry heat, or gamma sterilization and composition can be manufactured by aseptic processing. Alternatively, the long acting injectable composition of the present invention may be prepared using non-sterile cariprazine or its pharmaceutically acceptable salts thereof and the composition can then be sterilized using terminal sterilization process.

The long acting injectable composition of the present invention has a particle size D90 of cariprazine or its pharmaceutically acceptable salt between about 1 micron to about 200 microns. In one more embodiment, the long acting injectable composition of the present invention has a viscosity ranging from about 20 cps to about 200 cps. In a further embodiment, the long acting injectable composition of the present invention has a sediment height of about 30% to about 80%. In an embodiment, the redispersion time for the long acting injectable composition of the present invention is less than 1 minute, preferably about 5 seconds to about 50 seconds, more preferably about 10 seconds to about 30 seconds.

One embodiment is a long injectable aqueous suspension composition of the present intention having a particle size D90 between about 1 micron to about 200 microns, pH ranging from about 3 to about 8, viscosity ranging from about 20 cps to about 200 cps, sediment height between about 30% to about 80%, and redispersion time less than 1 min.

One more embodiment is a long injectable microsphere composition of the present intention having a particle size D90 between about 1 micron to about 200 microns, pH ranging from about 3 to about 8, viscosity ranging from about 20 cps to about 200 cps, sediment height between about 30% to about 80%, and redispersion time less than 1 min.

Another embodiment is a premeasured, prepackaged, premixed long acting injectable composition comprising cariprazine or its pharmaceutically acceptable salts thereof Preferably, the long acting injectable composition comprising cariprazine or its pharmaceutically acceptable salts thereof is a ready-to-use composition which does not require any mixing or dilution by the subject prior to administration. The long acting injectable composition comprising cariprazine or its pharmaceutically acceptable salts thereof may be administered for the treatment of psychotic disorders.

A. Aqueous suspension

The long acting injectable composition comprising cariprazine or its pharmaceutically acceptable salts thereof may be an aqueous suspension. In one embodiment, the long acting aqueous suspension comprises cariprazine hydrochloride. The long acting aqueous suspension composition may contain cariprazine or its pharmaceutically acceptable salts in micronized form. Alternatively, cariprazine or its pharmaceutically acceptable salts may be added in un-micronized form in the long acting aqueous suspension composition of the present invention. The long acting injectable composition of the present invention may contain cariprazine or its pharmaceutically acceptable salts thereof in amount of about 10 mg to about 400 mg.

Suitable excipients include, but are not limited to suspending agents, preservatives, buffers, tonicity adjusting agents and a vehicle.

Suitable suspending agents include, but are not limited to, sodium carboxymethyl cellulose, hydroxypropyl cellulose, carboxymethyl cellulose, hydroxypropylethyl cellulose, hydroxypropylmethyl cellulose, and polyvinylpyrrolidone, low molecular weight oligomers, natural products, and surfactants, such as cetyl pyridinium chloride, benzalkonium chloride, cetomacrogol emulsifying wax, sorbitan esters, polyoxyethylene alkyl ethers, polyoxyethylene castor oil derivatives, polyoxyethylene sorbitan fatty acid esters; polyethylene glycols, dodecyl trimethyl ammonium bromide, polyoxyethylene stearates, colloidal silicon dioxide, phosphates, sodium dodecylsulfate, carboxymethylcellulose tri calcium, hydroxypropyl celluloses, methylcellulose, hydroxyethylcellulose, hydroxypropylcellulose, hydroxypropylmethyl-cellulose phthalate, polyvinyl alcohol poloxamers, and charged phospholipids. The long acting injectable composition of the present invention may contain about 0.1 mg/ml to about 200 mg/ml of suspending agent. Preferably, the suspending agent is selected from polyethylene glycol, sorbitan ester, and polyoxyethylene sorbitan fatty acid esters.

Examples of preservatives, include, but are not limited to, benzyl alcohol, butylated hydroxytoluene (BHT), butylated hydroxyanisole (BHA), propyl gallate, methylparaben, propylparaben, tocopherols, and combinations thereof.

Suitable buffers, include, but are not limited to, phosphate, citrate, tartrate, succinate, phthalate, or acetate buffer. In a preferred embodiment, the buffer is selected from potassium dihydrogen phosphate, dipotassium hydrogen phosphate, sodium dihydrogen phosphate or sodium hydrogen phosphate, monobasic sodium phosphate and dibasic sodium phosphate. The long acting injectable composition of the present invention may contain about 1 mg/ml to about 40 mg/ml of buffer.

Example of tonicity agents, include, but are not limited to sodium chloride, potassium chloride, mannitol, sucrose, lactose, maltose, xylitol, glucose, sorbitol.

In one embodiment, the long acting injectable composition comprises cariprazine or its pharmaceutically acceptable salts thereof, a suspending agent, a preservative, a buffer, a tonicity adjusting agent and vehicle. In another embodiment, the long acting injectable composition comprises cariprazine or its pharmaceutically acceptable salts thereof, a suspending agent, a buffer, and vehicle. One embodiment relates to the use of the long acting injectable composition for treatment of psychotic disorders.

In one more embodiment, the long acting injectable composition comprises cariprazine or its pharmaceutically acceptable salts thereof, polyethylene glycol, monobasic sodium phosphate and dibasic sodium phosphate as buffer, polysorbate 80, sodium chloride and water for injection. One embodiment relates to the use of the long acting injectable composition for treatment of psychotic disorders.

In yet another embodiment, the long acting injectable composition comprises cariprazine or its pharmaceutically acceptable salts thereof, polyethylene glycol, monobasic sodium phosphate and dibasic sodium phosphate as buffer, sorbitan mono-oleate, sodium chloride and water for injection. One embodiment relates to the use of the long acting injectable composition for treatment of psychotic disorders.

In a further embodiment, the long acting injectable composition comprises cariprazine or its pharmaceutically acceptable salts thereof, polyethylene glycol, monobasic sodium phosphate and dibasic sodium phosphate as buffer, polysorbate 80, sorbitan mono-oleate, sodium chloride and water for injection. One embodiment relates to the use of the long acting injectable composition for treatment of psychotic disorders.

In one embodiment, the long acting injectable composition comprises cariprazine base, polyethylene glycol 4000, sorbitan monolaurate, polysorbate 20, citric acid monohydrate, sodium dihydrogen phosphate monohydrate, and water for injection.

In another embodiment, the long acting injectable composition comprises about 90 mg/ml to about 180 mg/ml cariprazine base, about 10 mg/ml to about 100 mg/ml polyethylene glycol 4000, about 0.1 mg/ml to about 3 mg/ml sorbitan monolaurate, about 1 mg/ml to about 20 mg/ml polysorbate 20, about 1 mg/ml to about 15 mg/ml citric acid monohydrate, about 2 mg/ml to about 20 mg/ml sodium dihydrogen phosphate monohydrate, and water for injection.

In a further embodiment, the long acting injectable composition comprises about 180 mg/ml cariprazine base, about 75 mg/ml polyethylene glycol 4000, about 1 mg/ml sorbitan monolaurate, about 5 mg/ml polysorbate 20, about 7.5 mg/ml citric acid monohydrate, about 6 mg/ml sodium dihydrogen phosphate monohydrate, and water for injection.

In one embodiment, the long acting injectable composition comprises cariprazine base, polyethylene glycol 4000, polysorbate 20, citric acid monohydrate, sodium dihydrogen phosphate monohydrate, and water for injection.

In one more embodiment, the long acting injectable composition comprises comprises about 90 mg/ml to about 180 mg/ml cariprazine base, about 10 mg/ml to about 100 mg/ml polyethylene glycol 4000, about 5 mg/ml to about 30 mg/ml polysorbate 20, about 1 mg/ml to about 15 mg/ml citric acid monohydrate, about 2 mg/ml to about 20 mg/ml sodium dihydrogen phosphate monohydrate, and water for injection.

In another embodiment, the long acting injectable composition comprises about 180 mg/ml cariprazine base, about 75 mg/ml polyethylene glycol 4000, about 5 mg/ml polysorbate 20, about 7.5 mg/ml citric acid monohydrate, about 6 mg/ml sodium dihydrogen phosphate monohydrate, and water for injection

In a further embodiment, the long acting injectable composition comprises comprises about 25 mg/ml to about 180 mg/ml cariprazine base, about 15 mg/ml to about 75 mg/ml polyethylene glycol, about 2 mg/ml to about 20 mg/ml monobasic sodium phosphate, about 2 mg/ml to about 20 mg/ml monobasic dibasic sodium phosphate, about 0.1 mg/ml to about 5 mg/ml polysorbate 80, about 0.1 mg/ml to about 5 mg/ml sorbitan mono-oleate, about 1 mg/ml to about 6 mg/ml sodium chloride and water for injection.

The long acting injectable composition of the present invention may contain pH between about 3 to about 8. The osmolality of the long acting injectable composition of the present invention may range from about 250 mOsm/kg to about 750 mOsm/kg, preferably about 300 mOsm/kg to about 500 mOsm/kg. The long acting injectable composition of the present invention may contain cariprazine or its pharmaceutically acceptable salt having a D90 from about 1 micron to about 200 microns. The viscosity of the long acting injectable composition may range from about 20 cps to about 200 cps. The long acting injectable composition of the present invention exhibit a redispersion time of about 5 seconds to about 50 seconds. The sediment height as observed for the long acting injectable composition could be between about 30% to about 80%

In one embodiment, the long acting injectable composition exhibits a D90 between about 1 micron to about 200 microns, pH ranging from about 3 to about 8, viscosity ranging from about 20 cps to about 200 cps, sediment height between about 30% to about 80%, and redispersion time of about 5 seconds to about 50 seconds.

In another embodiment, long acting injectable composition can be used for treatment of psychotic disorders.

The present invention will now be explained with reference to the following non-limiting examples.

### Example A 1

| Ingredient | Quantity (mg/mL) |
|---|---|
| Cariprazine (Base or Salt) | 25 to 180 |
| PEG 4000 | 15 to 75 |
| Monobasic sodium phosphate | 2 to 20 |
| Dibasic sodium phosphate | 2 to 20 |
| Polysorbate 80 | 0.1 to 5.0 |
| Sorbitan monooleate | 0.1 to 5.0 |
| Sodium Chloride | 1-6 |
| Water for injection | qs 1 mL |

Manufacturing Process:

Process 1:
1. Dissolve polyethylene glycol 4000, polysorbate 80, sorbitan monooleate, sodium chloride and buffering agents in water for injection (WFI) and filter through 0.2 μm filter.
2. Add Cariprazine (sterile) gradually to solution of step 1 under continuous stirring.
3. Stir the mixture for 2 hours using overhead stirring to ensure uniform mixing of Cariprazine in aqueous solution.
4. Measure pH of the suspension and if required adjust the pH between 3.0 to 7.0 using hydrochloric acid/sodium hydroxide solution.
5. Adjust the total volume of suspension using water for injection (WFI) and stir to ensure uniform mixing.
6. Fill the suspension in a suitable container closure system like vial/prefilled syringe.

Process 2:
1. Dissolve polyethylene glycol 4000, polysorbate 80, sorbitan monooleate, sodium chloride and buffering agents in water for injection (WFI).
2. Add Cariprazine gradually into solution of step 1 under continuous stirring.
3. Stir the mixture for 2 hours using overhead stirring to ensure uniform mixing of Cariprazine in aqueous solution.
4. Measure the pH of suspension and if required adjust the pH between 3.0 to 7.0 using hydrochloric acid/sodium hydroxide solution.
5. Adjust the total volume of suspension using water for injection (WFI) and stir to ensure uniform mixing.
6. Fill the suspension in a suitable container closure system like vial/prefilled syringe.
7. Terminally sterilize the finished product to get sterilized product.

Process 3:
1. Dissolve polyethylene glycol 4000, polysorbate 80, Sorbitan monooleate, sodium chloride and buffering agents in water for injection (WFI).
2. Add Cariprazine gradually into solution of step 1 under continuous stirring for 2 hours to ensure uniform mixing.
3. Mill the uniform mixture using media mill to get desire particle size.
4. Measure the pH of suspension and if required adjust the pH between 3.0 to 7.0 using hydrochloric acid/sodium hydroxide solution.
5. Adjust the total volume of suspension using water for injection (WFI) and stir to ensure uniform mixing.
6. Fill the suspension in a suitable container closure system like vial/prefilled syringe.
7. Aseptically fill the product or terminally sterilize via suitable process to get sterile finished product.

### Example A2

| Ingredients | Quantity (mg/mL) |
|---|---|
| Cariprazine base | 90 to 180 |
| Polyethylene Glycol 4000 | 10 to 100 |
| Sorbitan Monolaurate | 0.1 to 3.0 |
| Polysorbate 20 | 1 to 20 |
| Citric acid monohydrate | 1 to 15 |
| Sodium dihydrogen phosphate monohydrate | 2 to 20 |
| Sodium Hydroxide/HCL | qs to pH |
| Water for injection | qs to 1 mL |
| pH range | 3.0 to 9.0 |

Process of preparation:
1. Polyethylene glycol 4000, polysorbate 20, sorbitan monolaureate, citric acid monohydrate and sodium dihydrogen phosphate monohydrate were dissolved completely in water for injection (WFI) to prepare the aqueous phase.
2. Cariprazine was added gradually into solution of step 1 to ensure uniform mixing.
3. pH of suspension of step 2 was adjusted using hydrochloric acid/sodium hydroxide.
4. The suspension of step 3 was milled till a desired particle size of cariprazine was obtained.
5. The volume of suspension of step 4 was adjusted and stirred.
6. pH was measured and the suspension of step 5 was filled into an appropriate container.

### Example A3

| | Quantity |
|---|---|
| Ingredients | (mg/mL) |
| Cariprazine | 180 |
| Polyethylene Glycol 4000 | 75 |
| Sorbitan Monolaurate | 1 |
| Polysorbate 20 | 5 |
| Citric acid monohydrate | 7.5 |
| Sodium dihydrogen phosphate monohydrate | 6 |
| Sodium Hydroxide/HCL | q.s to pH |
| Water for injection | q.s to 1 mL |
| pH range | 3.0 to 9.0 |

Process of preparation:
1. Polyethylene glycol 4000, polysorbate 20, sorbitan monolaureate, citric acid monohydrate and sodium dihydrogen phosphate monohydrate were dissolved completely in water for injection (WFI) to prepare the aqueous phase.
2. Cariprazine was added gradually into solution of step 1 to ensure uniform mixing.
3. pH of suspension of step 2 was adjusted using hydrochloric acid/sodium hydroxide.
4. The suspension of step 3 was milled till a desired particle size of cariprazine was obtained.
5. The volume of suspension of step 4 was adjusted and stirred.
6. pH was measured and the suspension of step 5 was filled into an appropriate container.

Stability studies:

The stability of the long acting injectable composition A3 was analysed and it was observed that the redispersion time, assay, and impurities levels were within the acceptable limits throughout the study period.

The below table demonstrates the results of the stability studies.

| | | | 25° C./60% RH | | | 40° C./20% RH | | |
|---|---|---|---|---|---|---|---|---|
| Parameter | | Initial | 1 M | 3 M | 6 M | 1 M | 3 M | 6 M |
| Osmolarity | | 328 | 317 | 331 | 335 | 325 | 352 | 365 |
| Redispersion time (sec) | | ND* | 10 | 10 | 10 | 20 | 10 | 10 |
| % sediment | | ND* | 74.43 | 56.9 | 65.0 | 65.39 | 61.0 | 62.6 |
| Assay (%) | | 101.5 | 103.4 | 103.6 | 102.4 | 102.1 | 105.3 | 104.7 |
| Impurity | SMI | 0.14 | 0.16 | 0.13 | 0.17 | 0.16 | 0.2 | 0.26 |
| levels | Total | 0.25 | 0.38 | 0.24 | 0.28 | 0.41 | 0.32 | 0.84 |
| PSD | D90 | ND | 23.97 | 25.56 | 24.36 | 25.60 | 25.31 | 26.28 |

ND: Not Determined
SMI: Single maximum impurity

In-vitro drug release:

The In-vitro drug release of the long acting injectable composition A3 was evaluated, and below table represents the data for the in-vitro drug release. It was observed that the long acting injectable composition A3 was able to release over a period of about 96 hours.

| Time (hrs) | % drug release |
|---|---|
| 0 | 0 |
| 0.08 | 9 |
| 0.5 | 35 |
| 1 | 47 |
| 2 | 59 |
| 8 | 72 |
| 16 | 81 |
| 24 | 85 |
| 36 | 89 |
| 48 | 91 |
| 60 | 92 |
| 72 | 93 |
| 84 | 94 |
| 96 | 95 |

### Example A4

| Ingredients | Quantity (mg/mL) |
|---|---|
| Cariprazine base | 90 to 18 |
| Polyethylene Glycol 4000 | 10 to 100 |
| Polysorbate 20 | 5 to 30 |
| Citric acid monohydrate | 1 to 15 |
| Sodium dihydrogen phosphate monohydrate | 2 to 20 |
| Sodium Hydroxide/HCL | qs to pH |
| Water for injection | qs to 1 mL |
| pH range | 3.0 to 7.0 |

Process of preparation:
1. Polyethylene glycol 4000, polysorbate 20, citric acid monohydrate and sodium dihydrogen phosphate monohydrate were dissolved completely in water for injection (WFI) to prepare the aqueous phase.
2. Cariprazine was added gradually into solution of step 1 to ensure uniform mixing.
3. pH of suspension of step 2 was adjusted using hydrochloric acid/sodium hydroxide.
4. The suspension of step 3 was milled till a desired particle size of cariprazine was obtained.
5. The volume of suspension of step 4 was adjusted and stirred.
6. pH was measured and the suspension of step 5 was filled into an appropriate container.

### Example A5

| Ingredients | Quantity (mg/mL) |
|---|---|
| Cariprazine | 180 |
| Polyethylene Glycol 4000 | 75 |
| Polysorbate 20 | 5 |
| Citric acid monohydrate | 7.5 |
| Sodium dihydrogen phosphate monohydrate | 6 |
| Sodium Hydroxide/HCL | q.s to pH |
| Water for injection | q.s to 1 mL |
| pH range | 3.0 to 9.0 |

Process of preparation:
1. Polyethylene glycol 4000, polysorbate 20, citric acid monohydrate and sodium dihydrogen phosphate monohydrate were dissolved completely in water for injection (WFI) to prepare the aqueous phase.
2. Cariprazine was added gradually into solution of step 1 to ensure uniform mixing.
3. pH of suspension of step 2 was adjusted using hydrochloric acid/sodium hydroxide.
4. The suspension of step 3 was milled till a desired particle size of cariprazine was obtained.
5. The volume of suspension of step 4 was adjusted and stirred.
6. pH was measured and the suspension of step 5 was filled into an appropriate container.

Stability studies:

The stability of the long acting injectable composition A5 was analysed and it was observed that the redispersion time, assay, and impurities levels were within the acceptable limits throughout the study period.

The below table demonstrates the results of the stability studies.

| | | | 25 C./60% RH | | | 40 C./20% RH | | |
|---|---|---|---|---|---|---|---|---|
| Parameter | | Initial | 1 M | 3 M | 6 M | 1 M | 3 M | 6 M |
| Osmolarity | | 324 | 311 | 309 | 325 | 313 | 336 | 366 |
| Redispersion time | | — | 10 | 10 | 20 | 10 | 10 | 10 |
| (sec) | | | | | | | | |
| % Sediment height | | — | 72.91 | 62.81 | 70.63 | 65.73 | 60.50 | 73.03 |
| Assay | % LA | 105.2 | 102.3 | 104.9 | 102.1 | 102.5 | 99.4 | 104.1 |
| Impurity | SMI | 0.15 | 0.16 | 0.11 | 0.15 | 0.16 | 0.18 | 0.23 |
| levels | Total | 0.15 | 0.28 | 0.24 | 0.27 | 0.4 | 0.3 | 0.79 |
| PSD | D90 | ND | 23.58 | 24.09 | 25.62 | 25.55 | 26.31 | 26.65 |

ND: Not Determined

B. In-situ gel depot injection

The long acting injectable composition comprising cariprazine or its pharmaceutically acceptable salts thereof may be in the form of a depot composition which forms an in-situ gel upon injection. In one embodiment, the long acting injectable composition comprises cariprazine hydrochloride. The long acting injectable composition may contain cariprazine or its pharmaceutically acceptable salts in micronized form. Alternatively, cariprazine or its pharmaceutically acceptable salts may be added in un-micronized form in the long acting injectable composition of the present invention. The long acting injectable composition of the present invention may contain cariprazine or its pharmaceutically acceptable salts thereof in amount of about 10 mg to about 400 mg.

Suitable excipients include, but are not limited to rate controlling polymer, and solvents.

Suitable rate controlling polymers include, but are not limited to, cellulose derivatives, such as hydroxypropyl cellulose, hydroxyethyl cellulose, hydroxypropyl methyl cellulose, methylcellulose, sodium carboxymethyl cellulose, poly(lactic-co-glycolic acid) polymer, poly lactic acid, poly glycolic acid, sucrose acetate isobutyrate, triethyleneglycol poly(orthoester) polymer, vinyl polymers, polyoxyethylene- polyoxypropylene polymers or co-polymers (Pluronics®), polysaccharides such as glycosaminoglycans, agar, pectin, alginic acid, dextran, starch and chitosan, proteins, poly(ethyleneoxide), acrylamide polymers, polyhydroxy acids, polyanhydrides, polyorthoesters, polyamides, polycarbonates, polyalkylenes, polyalkylene glycols, polyethylene glycol, polyalkylene oxides, polyalkylene terepthalates, polyvinyl alcohols such as polyacrylic acid, polymethacrylic acid, polyvinyl pyrrolidone and polyvinyl alcohol, polyvinyl ethers, polyvinyl esters, polyvinyl halides, polyvinylpyrrolidone, polysiloxanes, polyvinyl acetates, polystyrene, polyurethanes, synthetic celluloses, polyacrylic acids, polybutyric acid, polyvaleric acid, poly(lactide-co- caprolactone), and copolymers, derivatives or mixtures thereof. In one embodiment, the long acting injectable composition of the present invention contain about 20 mg/ml to about 2000 mg/ml of rate controlling polymer.

Suitable organic solvents include, but are not limited to N-methyl pyrrolidone, dichloromethane, dichloroethane, chloroform, methyl acetate, ethyl acetate, acetonitrile, methanol, ethanol, iso-propyl alcohol and combinations thereof.

In an embodiment, the long acting injectable composition comprising cariprazine or its pharmaceutically acceptable salts thereof is a depot composition which forms an in-situ gel upon injection.

In another embodiment, the long acting injectable composition comprises cariprazine or its pharmaceutically acceptable salts thereof, atleast one rate controlling polymer, and solvents, wherein the said composition is an in-situ gelling composition.

In one embodiment, the long acting injectable composition of the present invention comprises cariprazine or its pharmaceutically acceptable salts thereof and the rate controlling polymer in a ratio of about 1:2 to about 1:6.

In another embodiment, the long acting injectable composition of the present invention comprises cariprazine or its pharmaceutically acceptable salts thereof and the rate controlling polymer in a ratio of about 1:3 to about 1:5

In a further embodiment, the long acting injectable composition comprises cariprazine or its pharmaceutically acceptable salts thereof, atleast one rate controlling polymer comprising poly(lactic-co-glycolic acid) polymer, sucrose acetate isobutyrate, triethyleneglycol poly(orthoester) polymer, and atleast one solvent comprising 2-N-methyl pyrollidone, dimethyl acetamide, dimethyl sulfoxide, ethanol, glyceryl triacetate, wherein the said composition is an in-situ gelling composition.

In one embodiment, the long acting injectable composition of the present invention comprises about 25 mg/ml to about 180 mg/ml cariprazine or its pharmaceutically acceptable salt thereof, about 25 mg/ml to about 500 mg/ml poly(lactic-co-glycolic acid) polymer and N-methyl pyrrolidone.

In another embodiment, the long acting injectable composition of the present invention comprises about 25 mg/ml to about 180 mg/ml cariprazine or its pharmaceutically acceptable salt thereof, about 25 mg/ml to about 500 mg/ml poly(lactic-co-glycolic acid) polymer, about 50 mg/ml to about 150 mg/ml polyethylene glycol and N-methyl pyrrolidone.

In one more embodiment, the long acting injectable composition of the present invention comprises about 90 mg/ml cariprazine or its pharmaceutically acceptable salt thereof, about 270 mg/ml poly(lactic-co-glycolic acid) polymer (PLGA 50:50) and N-methyl pyrrolidone.

In a further embodiment, the long acting injectable composition of the present invention comprises about 67.5 mg/ml cariprazine or its pharmaceutically acceptable salt thereof, about 337.5 mg/ml poly(lactic-co-glycolic acid) polymer (PLGA 50:50) and N-methyl pyrrolidone.

In one embodiment, the long acting injectable composition of the present invention comprises about 90 mg/ml cariprazine or its pharmaceutically acceptable salt thereof, about 270 mg/ml poly(lactic-co-glycolic acid) polymer (PLGA 95:5) and N-methyl pyrrolidone.

In one more embodiment, the long acting injectable composition of the present invention comprises about 90 mg/ml cariprazine or its pharmaceutically acceptable salt thereof, about 270 mg/ml poly(lactic-co-glycolic acid) polymer (PLGA 95:5), about 100 mg/ml polyethylene glycol (PEG400) and N-methyl pyrrolidone.

In a further embodiment, the long acting injectable composition of the present invention comprises about 90 mg/ml cariprazine or its pharmaceutically acceptable salt thereof, about 270 mg/ml poly(lactic-co-glycolic acid) polymer (PLGA 95:5), about 100 mg/ml polyethylene glycol (PEG4000) and N-methyl pyrrolidone.

In one embodiment, the long acting injectable composition of the present invention comprises about 90 mg/ml cariprazine or its pharmaceutically acceptable salt thereof, about 270 mg/ml poly(lactic-co-glycolic acid) polymer (PLGA 75:25) and N-methyl pyrrolidone.

In one more embodiment, the long acting injectable composition of the present invention comprises about 75 mg/ml cariprazine or its pharmaceutically acceptable salt thereof, about 225 mg/ml poly(lactic-co-glycolic acid) polymer (PLGA 50:50) and N-methyl pyrrolidone.

In another embodiment, the long acting injectable composition of the present invention comprises about 25 mg/ml to about 180 mg/ml cariprazine or its pharmaceutically acceptable salt thereof, about 50 mg/ml to about 1000 mg/ml sucrose acetate isobutyrate polymer and N-methyl pyrrolidone.

In a further embodiment, the long acting injectable composition of the present invention comprises about 25 mg/ml to about 180 mg/ml cariprazine or its pharmaceutically acceptable salt thereof, about 50 mg/ml to about 2000 mg/ml triethyleneglycol poly(orthoester) polymer and N-methyl pyrrolidone.

The present invention will now be explained with reference to the following non-limiting examples.

### Example B 1

| Ingredient | Quantity (mg/ml) |
|---|---|
| Cariprazine (Base or Salt) | 25 to 180 |
| Poly lactide co-glycolide | 50 to 1000 |
| Solvents | 50 to 2000 |

### Example B2

| Ingredient | Quantity (mg/ml) |
|---|---|
| Cariprazine (Base or Salt) | 25 to 180 |
| Sucrose acetate isobutyrate | 50 to 1000 |
| Solvents | 50 to 2000 |

### Example B3

| Ingredient | Quantity (mg/ml) |
|---|---|
| Cariprazine (Base or Salt) | 25 to 180 |
| Triethyleneglycol poly(orthoester) | 50 to 2000 |
| Solvents | 50 to 2000 |

Manufacturing process:
1. Dissolve polymer (Poly lactide co-glycolide/sucrose isobutyrate/Triethyleneglycol poly(orthoester) in suitable solvent under stirring to get clear solution.
2. Cariprazine (Base or Salt) was then added in polymer solution under stirring to get uniform product.
3. Fill the drug product in suitable container closure system like vial/prefilled syringe.
4. Aseptically filter or terminally sterilize via suitable process to get sterile finished product.

### Example B3

| | | PLGA | PLGA | PEG | PEG | PLGA | | |
|---|---|---|---|---|---|---|---|---|
| | Cariprazine | 50:50 | 95:5 | 400 | 4000 | 75:25 | | Drug:Polymer |
| Sr no. | mg/ml | mg/ml | mg/ml | mg/ml | mg/ml | mg/ml | NMP | ratio |
| B3.1 | 90 | 270 | — | — | — | — | q.s. | 1:3 |
| B3.2 | 67.5 | 337.5 | — | — | — | — | q.s | 1:5 |
| B3.3 | 90 | — | 270 | — | — | — | q.s | 1:3 |
| B3.4 | 90 | — | 270 | 100 | — | — | q.s | 1:3 |
| B3.5 | 90 | — | 270 | — | 100 | — | q.s | 1:3 |
| B3.6 | 90 | — | — | — | — | 270 | q.s | 1:3 |
| B3.7 | 75 | 225 | — | — | — | — | q.s | 1:3 |

Process:

Polymer such as PLGA/PEG was dissolved in organic solvent such as N-methyl pyrrolidone. Cariprazine base or salt was dissolved or dispersed in the polymer solution.

The below table demonstrates the analytical parameters for the compositions B3.1- B3.7.

| Sr no. | Assay (%) | Time for 50% drug release |
|---|---|---|
| B3.1 | 103.3 | 100 h |
| B3.2 | 100.8 | 240 h |
| B3.3 | 99.7 | 290 h |
| B3.4 | Not performed | Not performed |
| B3.5 | Not performed | Not performed |
| B3.6 | 95.2 | Not performed |
| B3.7 | Not performed | Not performed |

C. Implant

The long acting injectable composition comprising cariprazine or its pharmaceutically acceptable salts thereof may be in the form of an implant. In one embodiment, the long acting injectable composition comprises cariprazine hydrochloride. The long acting injectable composition may contain cariprazine or its pharmaceutically acceptable salts in micronized form. Alternatively, cariprazine or its pharmaceutically acceptable salts may be added in un-micronized form in the long acting injectable composition of the present invention.

Suitable excipients include, but are not limited to rate controlling polymer, and solvents.

Examples of rate controlling polymers include, but are not limited to, polylactide-co-glycolide, polylactic acid, polyglycolic acid, polycaprolactone, poly-anhydride, poly butyric ester-hydroxyl pentanoate copolymer, polypropylene glucosan, one or more of polylactic acid-polyglycol and combinations thereof.

Suitable organic solvents include, but are not limited to dichloromethane, chloroform, ethyl acetate, acetonitrile and combinations thereof. The organic solvent is evaporated during the manufacturing process and is not a part of the final composition.

In an embodiment, the long acting injectable composition comprising cariprazine or its pharmaceutically acceptable salts thereof is a depot composition which is in the form of an implant.

In another embodiment, the long acting injectable composition comprises cariprazine or its pharmaceutically acceptable salts thereof, and atleast one rate controlling polymer, wherein the said composition is an implant.

In yet another embodiment, the long acting injectable composition comprises cariprazine or its pharmaceutically acceptable salts thereof, and atleast one rate controlling polymer comprising poly(lactic-co-glycolic acid) polymer, polylactic acid polymers, wherein the said composition is an implant.

In one embodiment, the long acting injectable composition is an implant having a length of about 10 mm to about 30 mm and a diameter of about 0.5 mm to about 5 mm.

In a preferred embodiment, the long acting injectable composition is an implant having a length of about 15 mm and a diameter of about 1.5 mm.

The present invention will now be explained with reference to the following non-limiting examples.

### Example C₁

| Ingredient | Quantity (mg) |
|---|---|
| Cariprazine (Base or Salt) | 25 to 180 |
| poly(lactic-co-glycolic acid) polymer/ | 50 to 1000 |
| polylactic acid polymers | |
| Chloroform/ethanol | q.s. |

Manufacturing Process:

Process:
1. Dissolve Cariprazine (Base or Salt) and polymer in a suitable solvent mixture.
2. Lyophilize the solution to get uniform mixed dry powder of Cariprazine and polymer.
3. Extrude the dried powder through suitable means like using hot melt extrusion or co-extrusion or injection moulding or 3D printing to obtained cylindrical rod implant.
4. Place the implant in suitable injectable packaging system and terminally sterilize to render drug product sterile.

### Example C₂

| Ingredient | Quantity (mg) |
|---|---|
| Cariprazine (Base or Salt) | 25 to 180 |
| poly(lactic-co-glycolic acid) polymer/ | 50 to 1000 |
| polylactic acid polymers | |

Process:
1. Mix Cariprazine (Base or Salt) and polymer using suitable mill/blender to obtain uniform dry powder mixture.
2. Extrude the dried powder through suitable means like using hot melt extrusion or co-extrusion or injection moulding or 3D printing to obtained cylindrical rod implant.
3. Place the implant in suitable injectable packaging system and terminally sterilize to render drug product sterile.

The long acting injectable implant composition comprising cariprazine or its pharmaceutically acceptable salts thereof may be packaged in an appropriate container such as a syringe or an applicator containing the said implant, which can be directly injected without the need of further dilution or reconstitution.

D. Microsphere

The long acting injectable composition comprising cariprazine or its pharmaceutically acceptable salts thereof may be in the form of a microsphere. In one embodiment, the long acting injectable composition comprises cariprazine hydrochloride. The long acting injectable composition may contain cariprazine or its pharmaceutically acceptable salts in micronized form. Alternatively, cariprazine or its pharmaceutically acceptable salts may be added in un-micronized form in the long acting injectable composition of the present invention. The microsphere long acting injectable composition may be manufactured using single emulsification, double emulsification, phase-coacervation, cross-linking, and spray drying.

Suitable excipients include, but are not limited to rate controlling polymer, surfactants and solvents.

Suitable rate controlling polymer include, but are not limited to, poly(lactide-co-glycolide), polylactide, polyglycoli de, poly(lactide-co-glycolide)glucose, polycaprolactone, gelatin, hyaluronate and combination thereof. The long acting composition of the present invention may contain about 50 mg to about 1000 mg of the rate controlling polymer.

Suitable organic solvents include, but are not limited to dichloromethane, dichloroethane, chloroform, methyl acetate, ethyl acetate, acetonitrile, methanol, ethanol, iso-propyl alcohol and combinations thereof. The organic solvent is evaporated during the manufacturing process and is not a part of the final composition.

Suitable surfactants include, but are not limited to polyvinyl alcohol, sodium carboxymethyl cellulose, hydroxypropyl cellulose, carboxymethyl cellulose, hydroxypropylethyl cellulose, hydroxypropylmethyl cellulose, and polyvinylpyrrolidone, low molecular weight oligomers, natural products, and surfactants, such as cetyl pyridinium chloride, benzalkonium chloride, cetomacrogol emulsifying wax, sorbitan esters, polyoxyethylene alkyl ethers, polyoxyethylene castor oil derivatives, polyoxyethylene sorbitan fatty acid esters; polyethylene glycols, dodecyl trimethyl ammonium bromide, polyoxyethylene stearates, colloidal silicon dioxide, phosphates, sodium dodecylsulfate, carboxymethylcellulose tri calcium, hydroxypropyl celluloses, methylcellulose, hydroxyethylcellulose, hydroxypropylcellulose, hydroxypropylmethyl-cellulose phthalate, poloxamers, and charged phospholipids.

In an embodiment, the long acting injectable composition comprising cariprazine or its pharmaceutically acceptable salts thereof is a microsphere composition.

In another embodiment, the long acting injectable composition comprises cariprazine or its pharmaceutically acceptable salts thereof, and atleast one rate controlling polymer, wherein the said composition is in the form of microspheres.

In yet another embodiment, the long acting injectable composition comprises cariprazine or its pharmaceutically acceptable salts thereof, and atleast one rate controlling polymer comprising poly(lactic-co-glycolic acid) polymer, polylactic acid polymers, wherein the said composition is in the form of microspheres.

In one more embodiment, long acting injectable composition of the present invention comprises about 180 mg cariprazine or its pharmaceutically acceptable salts thereof and about 820 mg poly(lactic-co-glycolic acid) polymer.

In a further embodiment, long acting injectable composition of the present invention comprises about 160 mg cariprazine or its pharmaceutically acceptable salts thereof and about 840 mg poly (lactic-co-glycolic acid) polymer.

In one embodiment, long acting injectable composition of the present invention comprises about 120 mg cariprazine or its pharmaceutically acceptable salts thereof and about 880 mg poly (lactic-co-glycolic acid) polymer.

In one more embodiment, the long acting injectable microsphere composition may be supplied with a diluent. An exemplary embodiment of a diluent may contain carboxymethyl cellulose sodium, mannitol, Polysorbate 80 and water.

In one more embodiment, the long acting injectable composition is in the form of microspheres having a particle size D90 of about 1 micron to about 200 microns.

The present invention will now be explained with reference to the following non-limiting examples.

### Example D1

Sr.

| | | |
|---|---|---|
| No. | Components | Quantity per unit |

Microsphere manufacturing formula

| | | | |
|---|---|---|---|
| 1 | Cariprazine (Base or Salt) | 25-180 | mg |
| 2 | Polymer | 50-1000 | mg |
| 3 | Solvent | 150-1080 | mg |
| 4 | Polyvinyl alcohol | 25-400 | mg |

| | | |
|---|---|---|
| 5 | Water for injection | q.s. |

Diluent manufacturing formula

| | | | |
|---|---|---|---|
| 6 | Carboxymethylcellulose | 5-10 | mg |
| | Sodium | | |
| 7 | Mannitol | 50-100 | mg |
| 8 | Polysorbate 80 | 1-5 | mg |
| 9 | Water for injection | qs 1 | mL |

Manufacturing process:
1. Dissolve cariprazine base or salt and polymer in common organic solvent.
2. Simultaneously, dissolve the polyvinyl alcohol (PVA) in water or buffer in another vessel.
3. Add the cariprazine-polymer solution of step 1 to the aqueous PVA solution of step 2 under continuous stirring using in line homogenizer to form a stable emulsion.
4. Remove the organic solvent with or without dilution by WFI or buffer with the help of vacuum or nitrogen flush or heat.
5. Remove water and dry the product under vacuum with the help of heating procedure and separate the dried microspheres using an appropriate pharma CEP or PSL separator.
6. Mix the microsphere with mannitol solution to make slurry and fill the slurry of step 6 in a vial or pre-filled syringe and freeze dry using lyophilizer.
7. Alternatively, add mannitol and fill the powdered microsphere in primary packaging material such as vial or pre-filled syringe.
8. Prepare a diluent for microsphere suspension preparation by mixing carboxymethyl cellulose sodium, followed by polysorbate 80 and mannitol in water for injection. Stir the solution to get uniform product.
9. Fill diluent in a suitable container closure system such as vial or pre-filled syringe.

### Example D2

Sr.

| | | |
|---|---|---|
| No. | Components | Quantity per unit |

Microsphere manufacturing formula

| | | | |
|---|---|---|---|
| 1 | Cariprazine (Base or Salt) | 25-180 | mg |
| 2 | N-heptane | 0.1-100 | mL |
| 3 | Polymer | 75-540 | mg |
| 4 | Chloroform/Methanol | 0.1-10 | mL |
| 5 | Dichloromethane | 0.1-10 | mL |
| 6 | Polyvinyl alcohol | 25-400 | mg |
| 7 | Silicone oil | 0.1-100 | mL |
| 8 | Water for injection | qs to 100 | mL |

Diluent manufacturing formula

| | | | |
|---|---|---|---|
| 9 | Carboxymethylcellulose | 5-10 | mg |
| | Sodium | | |
| 10 | Mannitol | 50-100 | mg |
| 11 | Polysorbate 80 | 1-5 | mg |
| 12 | Water for injection | qs 1 | mL |

Manufacturing process:
1. Dissolve cariprazine base or salt in organic solvent under stirring to get clear solution.
2. Dissolve polymer in methanol under stirring to get clear solution in another vessel.
3. Add drug phase into polymer phase with continuous stirring and homogenization.
4. Add silicone oil to above solution for encapsulation of drug.
5. Add above mixture in emulsion prepared separately.
6. Emulsion can be prepared using heptane or hexane as oil phase and WFI or buffer of suitable pH as aqueous phase with the help of emulsifier like span.
7. Remove the organic solvent with or without dilution by WFI or buffer with the help of vacuum or nitrogen flush or heat.
8. Remove water and dry the product under vacuum with the help of heating procedure and separate the dried microspheres using an appropriate pharma CEP or PSL separator.
9. Mix the microsphere with mannitol solution to make slurry and fill the slurry of step 6 in a vial or pre-filled syringe and freeze dry using lyophilizer.
10. Alternatively, add mannitol and fill the powdered microsphere in primary packaging material such as vial or pre-filled syringe.
11. Prepare a diluent for microsphere suspension preparation by mixing carboxymethyl cellulose sodium, followed by polysorbate 80 and mannitol in water for injection. Stir the solution to get uniform product.
12. Fill diluent in a suitable container closure system such as vial or pre-filled syringe.

### Example D3

| Ingredient | | | | |
|---|---|---|---|---|
| (mg/mg of powder) | D3.1 | D3.2 | D3.3 | D3.4 |
| Cariprazine | 180 | 180 | 160 | 120 |
| Poly-lactic glycolic | 820 | 820 | 840 | 880 |
| acid | | | | |
| Dichloromethane* | 0.15 | NA | 0.7 mL | 0.53 mL |
| Chloroform* | NA | 0.4 mL | NA | NA |
| PVA solution %* | 0.25% | 0.25% | 0.25% | 0.125% |
| Microscopic | Free drug | Precipitation | Free drug | No drug |
| observations | crystals | of | crystals | crystals |
| | | formulation | | found |

*Solvents to be removed in process

Process:
1. Cariprazine or its salt and polymer such as PLGA was dissolved in common organic solvent solvents such as chloroform, and dichloromethane.
2. A solution of polyvinyl alcohol (PVA) was prepared by dissolving PVA in water in another vessel.
3. The cariprazine-polymer solution of step 2 was then then slowly added in aqueous PVA solution under continuous stirring using in line homogenizer.
4. The organic solvent from dispersion resulting from step 3 was evaporated at controlled temperature conditions.
5. After complete solvent evaporation, the solution of step 4 was filtered using filtration assembly.
6. The filtered microspheres were washed using water for injection to remove PVA from surface.
7. The washed microspheres were dried and then separated using pharma CEP or PSL separator and the dried microspheres were then filled in glass vial.

The analytical properties of the long acting injectable composition D3.4 is given in the table below:

| Assay (%) | | 98.4 |
|---|---|---|
| Particle size | D10 | 1.59 |
| distribution | D50 | 16.08 |
| (microns) | D90 | 99.42 |
| Related substance | SMI | 0.14 |
| (%) | Total | 0.24 |

E. Oily depot injection

The long acting injectable composition comprising cariprazine or its pharmaceutically acceptable salts thereof may be in the form of an oily depot. In one embodiment, the long acting injectable composition comprises cariprazine hydrochloride. The long acting injectable composition may contain cariprazine or its pharmaceutically acceptable salts in micronized form. Alternatively, cariprazine or its pharmaceutically acceptable salts may be added in un-micronized form in the long acting injectable composition of the present invention.

Suitable excipients include, but are not limited to oils and preservative.

Suitable oils include, but are not limited to, isopropyl myristate, ethyl oleate, castor oil, sesame oil, arachis oil, cottonseed oil, almond oil, olive oil, neatsfoot oil, maize oil, peanut oil, safflower oil, coconut oil, palm seed oil and combination thereof.

Examples of preservatives, include, but are not limited to, benzyl alcohol, butylated hydroxyltoluene (BHT), butylated hydroxyanisole (BHA), propyl gallate, methylparaben, propylparaben, tocopherols, and combinations thereof.

In an embodiment, the long acting injectable composition comprising cariprazine or its pharmaceutically acceptable salts thereof is an oil based depot composition.

In one more embodiment, the long acting injectable composition comprises cariprazine or its pharmaceutically acceptable salts thereof, atleast one oil, and a preservative.

In one embodiment, the long acting injectable composition comprises cariprazine or its pharmaceutically acceptable salts thereof, sesame oil and benzyl alcohol.

In yet another embodiment, the long acting injectable composition comprises cariprazine or its pharmaceutically acceptable salts thereof, cotton seed oil and benzyl alcohol.

In one more embodiment, the long acting injectable composition is in the form of oil based depot having a particle size D90 of about 1 micron to about 200 microns.

The present invention will now be explained with reference to the following non-limiting examples.

### Example E 1

| Ingredient | Quantity (mg) |
|---|---|
| Cariprazine (Base or Salt) | 25 to 180 |
| Benzyl alcohol | 5 to 100 |
| Sesame oil/ cotton seed oil | q.s. to 1 mL |

Manufacturing process:
1. Dissolve Cariprazine (base or salt) in suitable oil like Sesame oil/cotton seed oil or other pharmaceutically acceptable oil under stirring to get clear solution.
2. Add Cariprazine (Base or Salt) under stirring to get uniform product.
3. Fill the drug product in a suitable container closure system like vial/prefilled syringe.
4. Terminally sterilize via suitable process to get sterile finished product.

Kits/Devices

The long acting injectable composition comprising cariprazine or its pharmaceutically acceptable salts thereof of the present invention can be packaged in a suitable container.

Suitable containers include, but are not limited to vials, pre-filled injectable devices such as pre-filled syringe, auto-injectors, applicator etc. In one embodiment, the long acting injectable composition can be packaged as a kit comprising a container containing composition and a separate container containing diluent. The kit may further contain a device to aid reconstitution and administration.

One more embodiment is a kit comprising an injection device, instructions for using the device and the container containing the long acting injectable composition comprising cariprazine or its pharmaceutically acceptable salts thereof of the present invention. Yet another embodiment is a kit comprising an injection device, instructions for using the device, a container containing the long acting injectable composition comprising cariprazine or its pharmaceutically acceptable salts thereof of the present invention and a separate vial containing a diluent.

The long acting injectable composition comprising cariprazine or its pharmaceutically acceptable salts thereof may be packaged in an appropriate container such as a syringe containing the said implant, which can be directly injected without the need of further dilution or reconstitution.

Although the invention herein has been described with reference to embodiments, it is to be understood that these embodiments are merely illustrative of the principles and application of the present invention. It is therefore to be understood that numerous modifications may be made to the illustrative embodiments and that other arrangements may be devised without departing from the spirit and scope of the present invention as described.

1-51. (canceled)

52. A long acting injectable composition comprising cariprazine or its pharmaceutically acceptable salts thereof, a suspending agent, a buffer, and vehicle, wherein the injectable composition is in the form of an aqueous suspension.

53. The long acting injectable composition according to claim 52, wherein the composition comprises about 10 mg to about 400 mg cariprazine or its pharmaceutically acceptable salts thereof.

54. The long acting injectable composition according to claim 52, wherein the composition comprises about 0.1 mg/ml to about 200 mg/ml of suspending agent.

55. The long acting injectable composition according to claim 52, wherein the composition comprises about 1 mg/ml to about 40 mg/ml of buffer.

56. The long acting injectable composition according to claim 52, wherein the composition comprises cariprazine base, polyethylene glycol 4000, sorbitan monolaurate, polysorbate 20, citric acid monohydrate, sodium dihydrogen phosphate monohydrate, and water for injection.

57. The long acting injectable composition according to claim 52, wherein the composition comprises about 90 mg/ml to about 180 mg/ml cariprazine base, about 10 mg/ml to about 100 mg/ml polyethylene glycol 4000, about 0.1 mg/ml to about 3 mg/ml sorbitan monolaurate, about 1 mg/ml to about 20 mg/ml polysorbate 20, about 1 mg/ml to about 15 mg/ml citric acid monohydrate, about 2 mg/ml to about 20 mg/ml sodium dihydrogen phosphate monohydrate, and water for injection.

58. The long acting injectable composition according to claim 52, wherein the composition comprises cariprazine base, polyethylene glycol 4000, polysorbate 20, citric acid monohydrate, sodium dihydrogen phosphate monohydrate, and water for injection.

59. The long acting injectable composition according to claim 52, wherein the composition comprises about 90 mg/ml to about 180 mg/ml cariprazine base, about 10 mg/ml to about 100 mg/ml polyethylene glycol 4000, about 5 mg/ml to about 30 mg/ml polysorbate 20, about 1 mg/ml to about 15 mg/ml citric acid monohydrate, about 2 mg/ml to about 20 mg/ml sodium dihydrogen phosphate monohydrate, and water for injection.

60. The long acting injectable composition according to claim 52, wherein the composition comprises about 25 mg/ml to about 180 mg/ml cariprazine base, about 15 mg/ml to about 75 mg/ml polyethylene glycol, about 2 mg/ml to about 20 mg/ml monobasic sodium phosphate, about 2 mg/ml to about 20 mg/ml monobasic dibasic sodium phosphate, about 0.1 mg/ml to about 5 mg/ml polysorbate 80, about 0.1 mg/ml to about 5 mg/ml sorbitan mono-oleate, about 1 mg/ml to about 6 mg/ml sodium chloride and water for injection.

61. A long acting injectable composition comprising cariprazine or its pharmaceutically acceptable salts thereof, a rate controlling polymer, and solvent, wherein the injectable composition forms an in-situ gel upon injection.

62. The long acting injectable composition according to claim 61, wherein the composition comprises about 10 mg to about 400 mg cariprazine or its pharmaceutically acceptable salts thereof.

63. The long acting injectable composition according to claim 61, wherein the composition comprises cariprazine or its pharmaceutically acceptable salts thereof and the rate controlling polymer in a ratio of about 1:2 to about 1:6.

64. The long acting injectable composition according to claim 61, wherein the composition comprises about 20 mg/ml to about 2000 mg/ml of rate controlling polymer.

65. The long acting injectable composition according to claim 61, wherein the composition comprises about 25 mg/ml to about 180 mg/ml cariprazine or its pharmaceutically acceptable salt thereof, about 25 mg/ml to about 500 mg/ml poly(lactic-co-glycolic acid) polymer and N-methyl pyrrolidone.

66. The long acting injectable composition according to claim 61, wherein the composition comprises about 25 mg/ml to about 180 mg/ml cariprazine or its pharmaceutically acceptable salt thereof, about 25 mg/ml to about 500 mg/ml poly(lactic-co-glycolic acid) polymer, about 50 mg/ml to about 150 mg/ml polyethylene glycol and N-methyl pyrrolidone.

67. The long acting injectable composition according to claim 61, wherein the composition comprises about 25 mg/ml to about 180 mg/ml cariprazine or its pharmaceutically acceptable salt thereof, about 50 mg/ml to about 1000 mg/ml sucrose acetate isobutyrate polymer and N-methyl pyrrolidone.

68. The long acting injectable composition according to claim 61, wherein the composition comprises about 25 mg/ml to about 180 mg/ml cariprazine or its pharmaceutically acceptable salt thereof, about 50 mg/ml to about 2000 mg/ml triethyleneglycol poly(orthoester) polymer and N-methyl pyrrolidone.

69. A long acting injectable composition comprising cariprazine or its pharmaceutically acceptable salts thereof, cariprazine or its pharmaceutically acceptable salts thereof, a rate controlling polymer, and solvent, wherein the injectable composition is in the form of microsphere.

70. The long acting injectable composition according to claim 69, wherein the composition comprises about 10 mg to about 400 mg cariprazine or its pharmaceutically acceptable salts thereof.

71. The long acting injectable composition according to claim 69, wherein the composition comprises about 50 mg to about 1000 mg of the rate controlling polymer.

72. The long acting injectable composition according to claim 69, wherein the composition further comprises a diluent.
